(19) United States
(12) Patent Application Publication
(10) Pub. No.: US 2021/0379349 A1
(43) Pub. Date: Dec. 9, 2021

(54) SEMI-PERMANENT MAKEUP DEVICE

(71) Applicant: Miartek, Seongnam-si (KR)

(72) Inventors: Sang Rae KIM, Seoul (KR); Hyun Sook JIN, Seoul (KR)

(73) Assignee: Miartek, Seongnam-si (KR)

(21) Appl. No.: 17/334,832

(22) Filed: May 31, 2021

(30) Foreign Application Priority Data
Jun. 5, 2020 (KR) 10-2020-0068434

(51) Int. Cl. A61M 37/00

(52) U.S. Cl. CPC A61M 37/0076

(57) ABSTRACT

A tattoo device according to the present invention comprises a body including a hollow penetrating in the longitudinal direction; a compression ring coupled to the front end of the body; a chuck comprising a plurality of elastic support parts disposed in the body, a plurality of pressing parts extending from one ends of each elastic support parts and at least partly disposed in the compression ring, and a connection part combining the other ends of elastic support parts; a push member comprising a shaft part and a button part, the shaft part disposed in the body and coupled to the connection part of the chuck, the button part extending from the shaft part and at least partly protruding outside of rear end of the body; and a spring disposed in the body and providing an elastic force to push the push member toward the rear end of the body. A gap between the plurality of pressing parts may be widened when the push member is pushed toward the front end of the body.

The tattoo device according to the present invention can maintain a firmly fixed state of the tattoo needle without increasing the tightening strength of the gripper, can universally fix various types of tattoo needles. Further, because the overall configuration of the tattoo device according to the present invention is simple, it easy to manufacture. Also, it has the advantage of high ease of use.

### CROSS-REFERENCE TO RELATED APPLICATIONS

A claim for priority under 35 U.S.C. § 119 is made to Korean Patent Application No. 10-2020-0068434 filed on Jun. 5, 2020, in the Korean Intellectual Property Office, the entire contents of which are hereby incorporated by reference.

### TECHNICAL FIELD

The present invention relates to a semi-permanent makeup device capable of stably fixing various types of semi-permanent makeup needles.

### BACKGROUND ART

One exemplary way of semi-permanent makeup is tattoo which is engraving letters or patterns by injecting pigment into the skin or subcutaneous tissue using a tattoo needle to create fine scratches. One example of semi-permanent makeup device is a tattoo device. The tattoo devices are classified in various ways according to their operation method and form, largely according to the use of a power source, a so-called automatic method that causes the tattoo needle to vibrate using a motor, etc., and a finger method that is manually operated by the user.

In addition, tattoo needles applied to tattoo devices have various thicknesses and lengths. Tattoo needles can be classified into a bar-type in which only one needle is used, and a cartridge-type in which a plurality of needles are bound by a binder, depending on the combination of needles. Cartridge types are further divided into flat type, round type, etc. according to the form in which the needles are combined.

Conventional tattoo devices consist of a plurality of chucks for gripping tattoo needles and a body, and it is common to grip tattoo needles by adjusting the interval between each chuck.

Therefore, according to this prior art, the tattoo needle can be replaced because the tattoo needle is detachably fixed.

Such a tattoo device requires stability to prevent the gripped tattoo needle from shaking during the procedure, convenience to easily replace if necessary, and versatility in which various types of tattoo needles can be mounted.

On the other hand, tattoo needles have very low surface roughness so that other scars do not occur outside the point of penetration intended by the operator during the process of injecting the pigment into the skin. Conventional tattoo devices have the following problems because the chuck is made simple in shape.

### Problem 1

Since the contact surface between the tattoo needle and the chuck does not form much friction when the tattoo needle is gripped, the stability of the tattoo needle to prevent shaking depends only on the tightening strength of the chuck. In order to increase the tightening strength of the chuck, the chuck and the body must be screwed together. Therefore, there are restrictions on the association configuration.

### Problem 2

If the tightening strength of the chuck is excessively increased, morphological deformation may occur in the tattoo needle. In particular, the risk of morphological deformation increases in the case of a cartridge type tattoo needle that is structurally weak.

### Problem 3

As the tightening strength of the chuck increases, the operation for releasing the tightening state becomes relatively inconvenient, so the convenience of use decreases.

### DETAILED DESCRIPTION OF THE INVENTION

### Technical Problem

The present invention was conceived to solve the problems of the prior art described above, and is to provide a tattoo device capable of firmly fixing several types of tattoo needles without increasing the tightening strength of the chuck.

### Technical Solution

A tattoo device according to the present invention comprises a body including a hollow penetrating in the longitudinal direction; a compression ring coupled to the front end of the body; a chuck comprising a plurality of elastic support parts disposed in the body, a plurality of pressing parts extending from one ends of each elastic support parts and at least partly disposed in the compression ring, and a connection part combining the other ends of elastic support parts; a push member comprising a shaft part and a button part, the shaft part disposed in the body and coupled to the connection part of the chuck, the button part extending from the shaft part and at least partly protruding outside of rear end of the body; and a spring disposed in the body and providing an elastic force to push the push member toward the rear end of the body. A gap between the plurality of pressing parts may be widened when the push member is pushed toward the front end of the body.

In the tattoo device according to the present invention, the connection part may be a non-segmented integral part of the parent body of the plurality of elastic support parts.

In the tattoo device according to the present invention, the connection part may include a connection member combining the plurality of elastic support parts.

In the tattoo device according to the present invention, the body and the compression ring may be coupled by screw connection, and the connection part and the shaft part may be coupled by screw connection.

In the tattoo device according to the present invention, the thickness of each elastic support part may be smaller than the thickness of each pressing part.

In the tattoo device according to the present invention, the thickness of each of the elastic support part may be between 0.2 mm and 0.3 mm.

In the tattoo device according to the present invention, the inclination angle formed between outer surface of the elastic support part and the outer surface of pressing part may be between 150 and 175 degrees.

In the tattoo device according to the present invention, the inner circumferential surface of the compression ring may have a tapered shape, and the outer circumferential surface formed in a state in which the plurality of pressing parts may be in close contact with each other has a shape corresponding to the tapered shape of the compression ring.

In the tattoo device according to the present invention, wherein occlusal surfaces of the pressing parts of the chuck facing each other may be formed in a serrated shape by forming a plurality of longitudinal or transverse grooves.

In the tattoo device according to the present invention, the body may include a locking projection protruding toward the center of the hollow, and the push member may include a stepped portion formed by making the diameter of the button part larger than the diameter of the shaft part, and the spring may be disposed between the locking projection and the stepped portion.

In the tattoo device according to the present invention, a groove may be formed on the front end the shaft part of the push member.

In the tattoo device according to the present invention, the spring may be configured to be elastically compressed by less than 15% when the push member is released.

In the tattoo device according to the present invention, the spring may be configured to be elastically compressed by more than 20% when the push member is pushed into the body.

### Advantageous Effects

The tattoo device according to the present invention can maintain a firmly fixed state of the tattoo needle without increasing the tightening strength of the gripper, can universally fix various types of tattoo needles. Further, because the overall configuration of the tattoo device according to the present invention is simple, it easy to manufacture. Also, it has the advantage of high ease of use.

### BRIEF DESCRIPTION OF THE DRAWINGS

FIG. 1 is a view showing the appearance of a tattoo device according to the present invention.

FIG. 2 is an exploded view showing the configuration of a tattoo device according to the present invention.

FIG. 3 is a view showing the chuck of the tattoo device according to the present invention.

FIG. 4 is a view showing the inclination angle of the pressing part and the elastic support part of the chuck of the tattoo device according to the present invention.

FIG. 5 is a view showing a cross-section of a tattoo device according to the present invention.

FIGS. 6A and 6B are views showing the process of fixing a tattoo needle (bar type) to the tattoo device according to the present invention.

FIG. 7 is a partially enlarged view showing a state in which a tattoo needle (bar type) is fixed to the tattoo device according to the present invention.

FIG. 8 is a view showing a state in which a tattoo needle (cartridge type) is fixed to the tattoo device according to the present invention.

FIG. 9 is a partially enlarged view showing a state in which a tattoo needle (cartridge type) is fixed to the tattoo device according to the present invention.

### BEST MODE

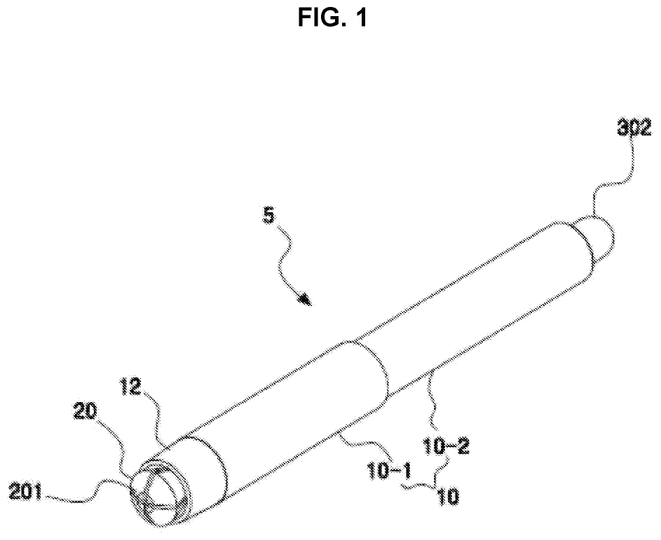

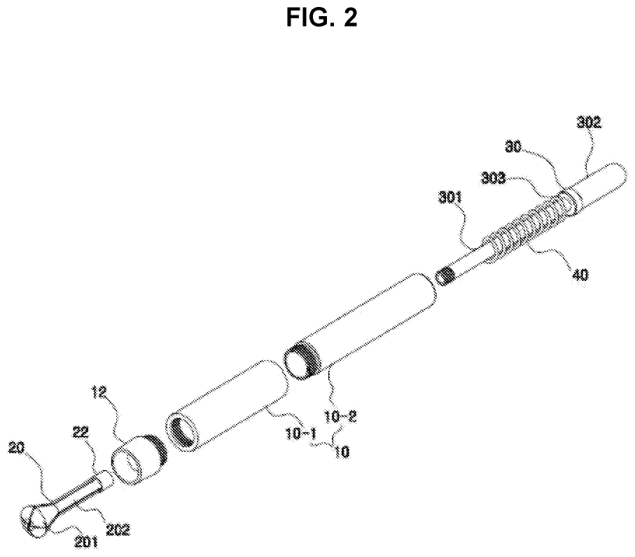

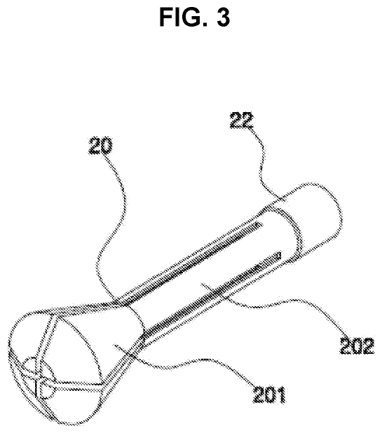

Hereinafter, specific details for implementing the present invention will be described in detail with reference to FIGS. 1 to 9.

As shown in FIGS. 1 to 5, the tattoo device 5 according to the present invention includes a body 10 having a hollow 10a penetrating in the longitudinal direction, and a compression ring 12 coupled to the front end of the body 10, a chuck 20 for gripping the tattoo needle according to the state of protrusion from the compression ring 12, a push member 30 providing an operating pressure to the chuck 20 in the direction of the tip of the body 10, and a spring 40 that provides an elastic force to the push member 30.

The body 10 may be made of a structure in which the front half part 10-1 and the rear half part 10-2, which are configured separately in order to improve assembleability, are screwed together, or alternatively, the body 10 may be made of a single entity (not shown).

The chuck 20 includes a plurality of elastic support parts 202, a plurality of pressing parts 201 extending from one end (front end) of each of the elastic support parts 202, and a connection part 22 combining the other ends (rear end) of the plurality of elastic support parts 202. The pressing parts 201 and the elastic support parts 202 of the chuck 20 are in a form in which the cylindrical parent body is segmented in the longitudinal direction. In a case that the elastic support parts 202 are partly segmented, the connection part 22 may be a a non-segmented integral part of the parent body. In a case that the elastic support part 202 are entirely segmented, the connection part 22 may include a separate connection member (not shown) combining the plurality of elastic support parts 202. The thickness of the connection part 22 in the radial direction may be same to or larger than the thickness of the parent body of the elastic support part 202 in the radial direction.

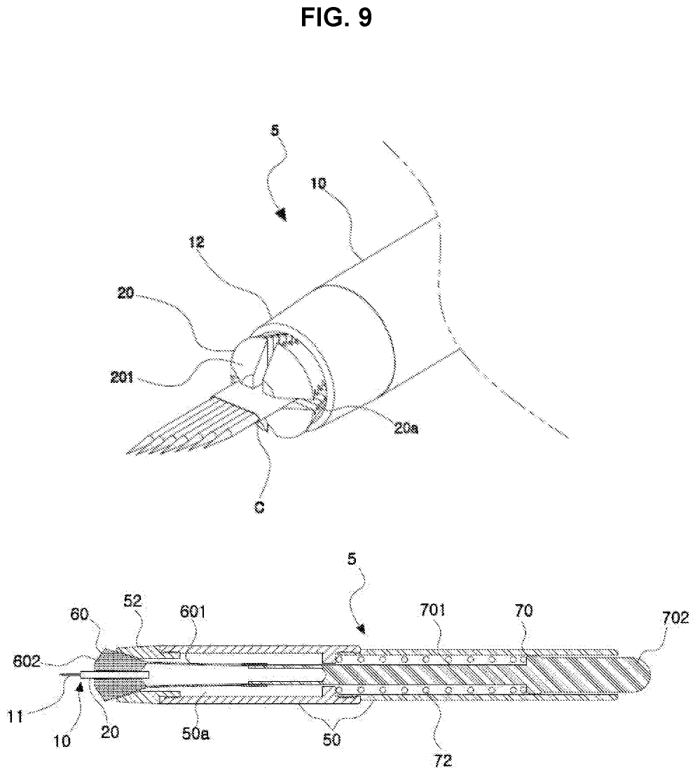

The occlusal surfaces of the pressing parts 201 of the chuck 20 facing each other may be formed in a serrated shape by forming a plurality of longitudinal or transverse grooves 20a. See FIG. 9.

The thickness of each of the elastic support part 202 is smaller than the thickness of each of the pressing part 201. Preferably, the thickness of each of the elastic support part 202 is between 0.2 mm and 0.3 mm.

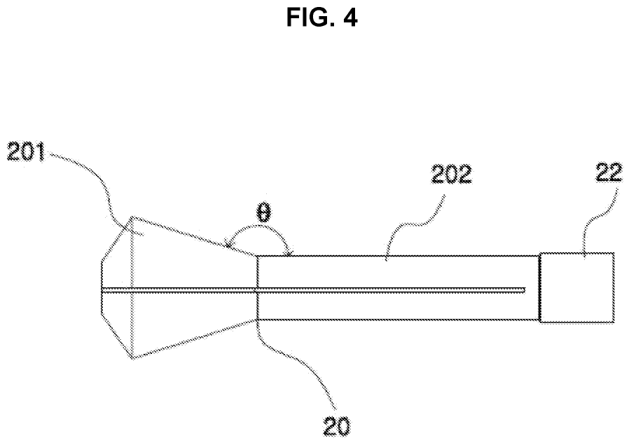

In addition, it is preferable that the inclination angle 9 formed between outer surface of the elastic support part 202 and the outer surface of pressing part 201 is between 150 and 175 degrees as shown in FIG. 4.

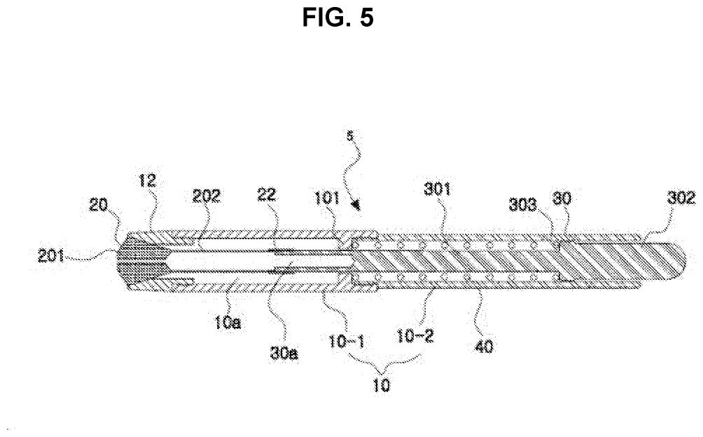

The connection part 22 of the chuck 20 is coupled with the shaft part 301 of the push member 30. For example, the connection part 22 may be coupled with the shaft part 301 by screw connection. More specifically, the connection part 22 may function as nut with its thread formed on its inner circumferential surface formed by penetrating hole and the shaft part 301 may function as bolt with its thread formed on its outer circumferential surface as shown in FIG. 5. Alternatively, the connection part 22 may function as bolt and the shaft part 301 may function as nut (not shown).

At least a portion of the elastic support part 202 of the chuck 20 and the connection part 22 are disposed in the hollow 10a in the body 10. At least portions of the pressing parts 201 are disposed in the compression ring 12 and at least portions of the pressing parts 201 protrude outside the compression ring 12.

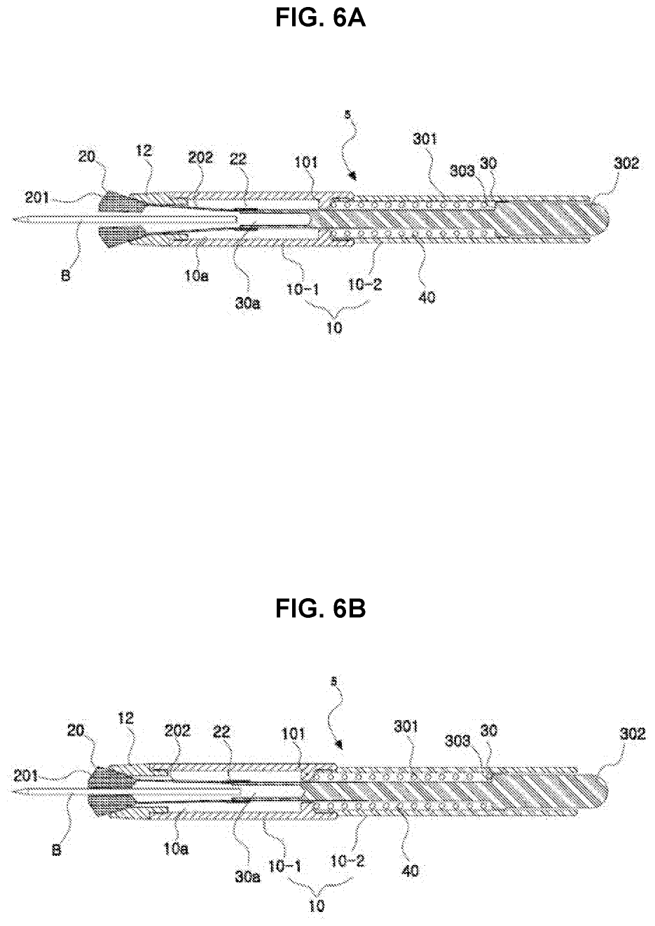

The plurality of pressing parts 201 of the chuck 20 are maintained in a state spaced apart from each other by the elastic support parts 202 in a state in which no force is applied. See FIG. 6a.

The inner circumferential surface of the compression ring 12 has a tapered shape, and the outer circumferential surface formed in a state in which the plurality of pressing parts 201 are in close contact with each other has a shape corresponding to the tapered shape of the compression ring 12.

The push member 30 is disposed inside the body 10 and includes a shaft part 301 coupled to connection part 22 of the chuck 20 and a button part 302 protruding outward in the rear end direction of the body 10.

Referring to FIG. 5, the body 10 includes a locking projection 101 protruding toward the center of the hollow 10a. The push member 30 includes a stepped portion 303 formed by making the diameter of the button part 302 larger than the diameter of the shaft part 301. The spring 40 is disposed between the locking projection 101 and the stepped portion 303.

As shown in FIG. 5, a groove 30a may be formed on the front end the shaft part 301 of the push member 30. The groove 30a is connected with, i.e., air-communicating with the penetrating hole of the connection part 22 and the gap between the elastic support parts 202 of the chuck 20.

The spring 40 is configured to be elastically compressed by 15% or less from the natural state length (length without external pressure) to maintain a preloaded state when the user does not press the button part 302 of the push member 30. In this state, the preloaded spring 40 has elastic force to push the push member 30 toward the rear end of the body 10.

However, the push member 30 stops because the pressing parts 201 are stucked by the compression ring 12 and the chuck 20 and the push member 30 are coupled. When the push member 30 is pushed into the body 10, the spring 40 is elastically compressed by more than 15%, or more preferably, more than 20% from the natural state length.

There is a gap between the inner circumferential surface of the body 10 and the elastic support parts 202 so that the elastic support parts 202 do not contact the inner circumferential surface of the body 10 when the elastic support parts 202 are operated.

Hereinafter, an operation of gripping a bar-type tattoo needle by the tattoo device according to the present invention will be described. Bar-type tattoo needle is long and thick, and consist of only one individual needle.

First, referring to FIG. 5, in the natural state in which the user does not press the button part 302 of the push member 30, because the spring 40 is preloaded as described above, the chuck 20 is lightly pulled toward inside the body 10 and thus the chuck 20 does not protrude to the outside of the compression ring 12.

Then, when the user presses the button part 302 of the push member 30, the chuck 20 is pushed by the push member 30 to protrude from the compression ring 12, and the gap between each pressing part 201 of the chuck 20 is elastically expanded.

At this time, the spring 40 is elastically compressed by the stepped portion 303 and the locking projection 101 as the gap between the stepped portion 303 and the locking projection 101 is narrowed.

When the gap between the compression units 201 is expanded, the tattoo needle B is inserted between the compression units 201, and then, when the push member 30 is released, the spring 40 is elastically restored. Accordingly, the push member 30 is returned to its original position, and the chuck 20 is also pulled toward the inside of the compression ring 12 by the push member 30.

Accordingly, the outer circumferential surface of each pressing part 201 of the chuck 20 interferes with inner circumferential surface of the compression ring 12, so that the gap between the pressing parts 201 is narrowed, and the tattoo needle B is gripped by the pressing parts 201. See FIGS. 6a and 6b.

As shown in FIG. 4, in the characteristic that the inclination angle 9 formed by the outer surface of pressing part 201 of the chuck 20 and the outer surface of the elastic support part 202 is between 150 degrees and 175 degrees, the so-called interference pressure that the pressing parts 201 receives from the compression ring 12 is optimized so that the tattoo needle may be gripped firmly.

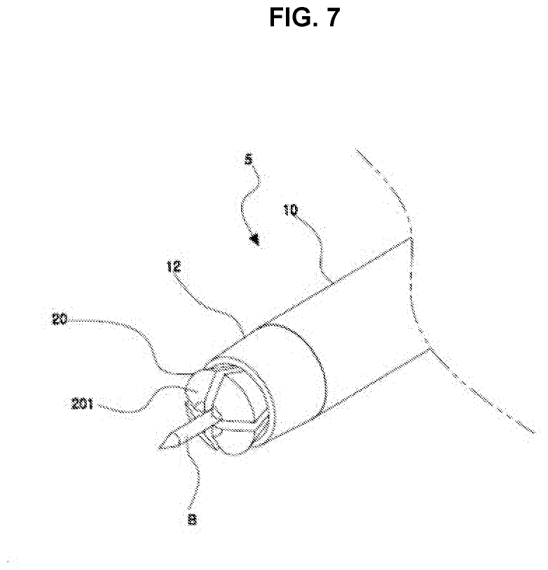

In addition, as shown in FIG. 7, since the elastic support part 202, which is thinner than the pressing part 201, can be flexibly curved, the pressing part 201 can be in surface contact with the tattoo needle B. As a result, the tattoo needle B can be gripped more stably.

When the inclination angle 9 is less than 150 degrees, the interference pressure is not formed sufficiently because the inclination of the pressing part 201 is excessively large. When the inclination angle is more than 175 degrees, there is a disadvantage that the operating interval of the chuck 20 must be increased in order to form an appropriate interference pressure.

In addition, as shown in FIG. 6b, despite the bar-type tattoo needle B is long and thick, in the gripped state, its rear end passes through between elastic support parts 202 and the connection part 22, and inserted into the groove 30a of the shaft part 301, so as not to interfere with each component.

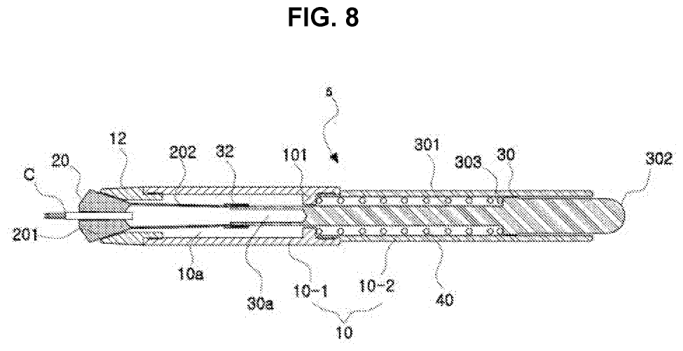

On the other hand, as shown in FIGS. 8 and 9, the cartridge type tattoo needle C has a form in which a plurality of needles are bound by a binder. The binder is gripped by the pressing part 201. The binder may be made of a plastic material, and thus may be softer than metal while its surface may be rough.

As shown in FIGS. 8 and 9, a longitudinal or transverse groove 20a is formed on the occlusal surface of the pressing part 201, so that the cartridge-type tattoo needle C does not slide and fall out and more stable gripping state may be maintained.

The tattoo device according to the present invention can be assembled in the following order.

1) The shaft part 301 of the push member 30 is inserted into the spring 40.

2) The push member 30 and the spring 40 are inserted into the front half part 10-1 of the body 10.

3) The chuck 20 is inserted into the compression ring 12.

4) The push member 30 and the chuck 20 are coupled.

5) The front half part 10-1 of the body 10 and the compression ring 12 are combined.

6) The front half part 10-1 and the rear half part 10-12 of the body 10 are combined.

The push member 30 and the chuck 20, the front half part 10-1 and the compression ring 12 of the body 10, and the front half part 10-1 and the rear half part 10-2 of the body 10 can be coupled by screw connection, and the order of connection can be changed.

[Reference Signs and Numerals]

| | |
|---|---|
| 5: taatoo device | 10: body |
| 12: compression ring | 20: chuck |
| 201: pressing part | 202: elastic support part |
| 30: push member | 30a: groove |
| 301: shaft part | 302: button part |
| 40: spring | |

What is claimed is:

1. A tattoo device comprising:
a body including a hollow penetrating in the longitudinal direction;
a compression ring coupled to the front end of the body;
a chuck comprising a plurality of elastic support parts disposed in the body, a plurality of pressing parts extending from one ends of each elastic support parts and at least partly disposed in the compression ring, and a connection part combining the other ends of elastic support parts;
a push member comprising a shaft part and a button part, the shaft part disposed in the body and coupled to the connection part of the chuck, the button part extending from the shaft part and at least partly protruding outside of rear end of the body; and
a spring disposed in the body and providing an elastic force to push the push member toward the rear end of the body,
wherein a gap between the plurality of pressing parts is widened when the push member is pushed toward the front end of the body.

2. The tattoo device of claim 1,
wherein the connection part is a non-segmented integral part of the parent body of the plurality of elastic support parts.

3. The tattoo device of claim 1,
wherein the connection part includes a connection member combining the plurality of elastic support parts.

4. The tattoo device of claim 1,
wherein the body and the compression ring are coupled by screw connection, and
wherein the connection part and the shaft part are coupled by screw connection.

5. The tattoo device of claim 1,
wherein the thickness of each elastic support part is smaller than the thickness of each pressing part.

6. The tattoo device of claim 5,
wherein the thickness of each of the elastic support part is between 0.2 mm and 0.3 mm.

7. The tattoo device according to the claim 1,
wherein the inclination angle formed between outer surface of the elastic support part and the outer surface of pressing part is between 150 and 175 degrees.

8. The tattoo device according to the claim 1,
wherein the inner circumferential surface of the compression ring has a tapered shape, and the outer circumferential surface formed in a state in which the plurality of pressing parts are in close contact with each other has a shape corresponding to the tapered shape of the compression ring.

9. The tattoo device according to the claim 1,
wherein occlusal surfaces of the pressing parts of the chuck facing each other are formed in a serrated shape by forming a plurality of longitudinal or transverse grooves.

10. The tattoo device according to the claim 1,
the body includes a locking projection protruding toward the center of the hollow, and
the push member includes a stepped portion formed by making the diameter of the button part larger than the diameter of the shaft part, and
the spring is disposed between the locking projection and the stepped portion.

11. The tattoo device according to the claim 1,
wherein a groove is formed on the front end the shaft part of the push member.

12. The tattoo device according to the claim 1,
wherein the spring is configured to be elastically compressed by less than 15% when the push member is released.

13. The tattoo device according to the claim 1,
wherein the spring is configured to be elastically compressed by more than 20% when the push member is pushed into the body.
